(19) United States
(12) Patent Application Publication
(10) Pub. No.: US 2007/0016226 A1
(43) Pub. Date: Jan. 18, 2007

(54) Implanter

(75) Inventors: Neil Campbell, Eden, MD (US); Chinsoo Park, Salisbury, MD (US)

Correspondence Address: STROOCK & STROOCK & LAVAN LLP, 180 MAIDEN LANE, NEW YORK, NY, 10038, US

(73) Assignee: Bio Medic Data Systems, Inc.

(21) Appl. No.: 11/202,753

(22) Filed: Aug. 11, 2005

Related U.S. Application Data
(60) Provisional application No. 60/699,115, filed on Jul. 13, 2005.

(51) Int. Cl. A61B 17/00

(52) U.S. Cl. 606/116

(57) ABSTRACT

An apparatus for implanting a marker in an animal is provided, which comprises a cannula having a sharp distal end and a proximal end, a housing comprising an aperture through which adhesive can be inserted into an adhesive receiving space, and a pushrod, wherein the cannula and the housing are formed physically independent of each other and assembled thereafter. A method of producing an apparatus for implanting a marker in an animal is also provided, wherein a cannula having a sharp distal end is formed, a housing having an adhesive receiving space is injection molded, and the cannula is inserted into the housing and affixed thereto by filling the adhesive receiving space with and effective amount of adhesive.

### CROSS-REFERENCE TO RELATED APPLICATIONS

This application claims the benefit of U.S. Provisional Application Ser. No. 60/699,115, filed on Jul. 13, 2005, entitled “IMPLANTER”, hereby incorporated by reference.

### BACKGROUND OF THE INVENTION

This invention relates, in general, to an apparatus for implanting a marker, in an animal and, in particular, an identification or sensory marker in a laboratory animal, and methods of producing an apparatus for implanting a marker in an animal.

Various markers can be implanted in an animal, according to the purpose being served. For example, identification markers can include transponders containing information regarding the identity of the animal, and can provide the stored information when scanned by a reader or at predetermined intervals. Another example of markers include markers that can collect information from the animal. For example, the marker can comprise mechanisms to collect physiological data regarding the animal, such as its temperature. Such a marker can be beneficial to monitor the health of the animal. A marker can comprise both of these features as well.

Markers typically comprise a capsule housing the necessary components and is implanted utilizing an apparatus having a sharp cannula. A marker can either be inserted into the cannula during manufacture or by the user prior to implantation. The user typically inserts the sharp end of the cannula containing the marker into the animal and then inserts a pushrod into the opposite end of the cannula, thereby pushing the marker with the pushrod out of the sharp end of the cannula and into the animal.

A currently available apparatus for implanting makers is typically insert molded, wherein a cannula is inserted into a mold for the housing, and the housing is molded around the cannula. However, during the insert molding process of conventional methods, the distal end of the cannula can be damaged, for example, while being inserted into the mold or being removed from the mold. The distal end of the cannula is typically angled and has a tip sufficiently sharp enough to pierce the skin of the animal to initiate implantation of the marker. Therefore, damaging the sharp distal tip can be detrimental to the performance of the implanting apparatus and thus can result in numerous products or components thereof being discarded during production.

In light of shortcomings described above, it is desirable to provide an implantation apparatus and methods of producing and/or assembling an implantation apparatus for implanting a marker that can overcome the discrepancies of the conventional implantation apparatus and methods of production thereof known in the art.

### SUMMARY

Generally speaking, the present invention is directed toward an apparatus for implanting a marker in an animal and methods of producing an apparatus for implanting a marker. It is to be understood that the term “producing” includes forming, assembling, combining, etc., and is not limited to any particular process or manner. Additionally, it is to be understood that the term “animal” includes mammals, such as humans, rodents, etc., as well as fish, reptiles, etc. More specifically, in accordance with an embodiment of the invention, an apparatus for implanting a marker in an animal comprises a cannula and a housing that can be assembled after the independent production of the housing and the cannula components.

In accordance with one embodiment of the invention, an implanting apparatus comprises a cannula having a sharp distal end and a blunt proximal end and a housing, wherein the housing is molded independently of the cannula. The blunt proximal end of the cannula can be inserted into the distal end of the housing, which preferably comprises an adhesive insertion portion. An effective amount of adhesive can thereafter be inserted into the adhesive insertion portion, thereby securing the cannula in place with respect to the housing.

Accordingly, it is an object of the invention to provide a method for producing an apparatus for implanting a marker having a housing that is produced independently of a cannula and combined thereafter.

Additionally, it is an object of the invention to provide a method for assembling an apparatus for implantation having a housing that is injection molded independently of a cannula and combined thereafter.

Other objects and features of the present invention will become apparent from the following detailed description, considered in conjunction with the accompanying drawing figure. It is to be understood, however, that the drawings are designed solely for the purpose of illustration and not as a definition of the limits of the invention, for which reference should be made to the appended claims.

### BRIEF DESCRIPTION OF THE FIGURES

For a fuller understanding of the invention, reference is had to the following description, taken in connection with the accompanying drawings, in which:

FIG. 1 is a perspective view of an apparatus for implanting a marker constructed in accordance with an embodiment of the invention;

FIG. 2 is an exploded perspective view of an apparatus for implanting a marker constructed in accordance with an embodiment of the invention with a protective cover and a marker;

FIG. 3 is a top view of a housing of an apparatus for implanting a marker constructed in accordance with an embodiment of the invention;

FIG. 4 is a side view of the housing of FIG. 3;

FIG. 5 is a cross-sectional view of the housing of FIG. 3 taken along line A-A;

FIG. 6 is a cross-sectional view of the housing of FIG. 3 taken along line B-B;

FIG. 7 is a side view of the housing of FIG. 3;

FIG. 8 is a magnified view of area A of FIG. 3;

FIG. 9 is a magnified view of area B of FIG. 3;

FIG. 10 is a bottom view of the housing of FIG. 3;

FIG. 11 is a top view of a pushrod of an apparatus for implanting a marker constructed in accordance with an embodiment of the invention;

FIG. 12 is a side view of the pushrod of FIG. 11;

FIG. 13 is a side view of the pushrod of FIG. 11;

FIG. 14 is a bottom view of the pushrod of FIG. 11;

FIG. 15 is a magnified view of area C of FIG. 14;

FIG. 16 is a top view of a protective cover of an apparatus for implanting a marker constructed in accordance with an embodiment of the invention; and

FIG. 17 is a side view of the protective cover of FIG. 16.

### DETAILED DESCRIPTION

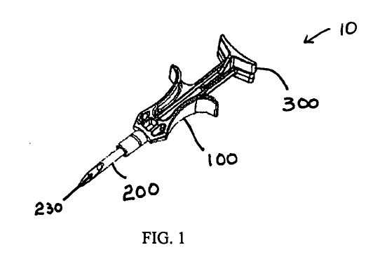

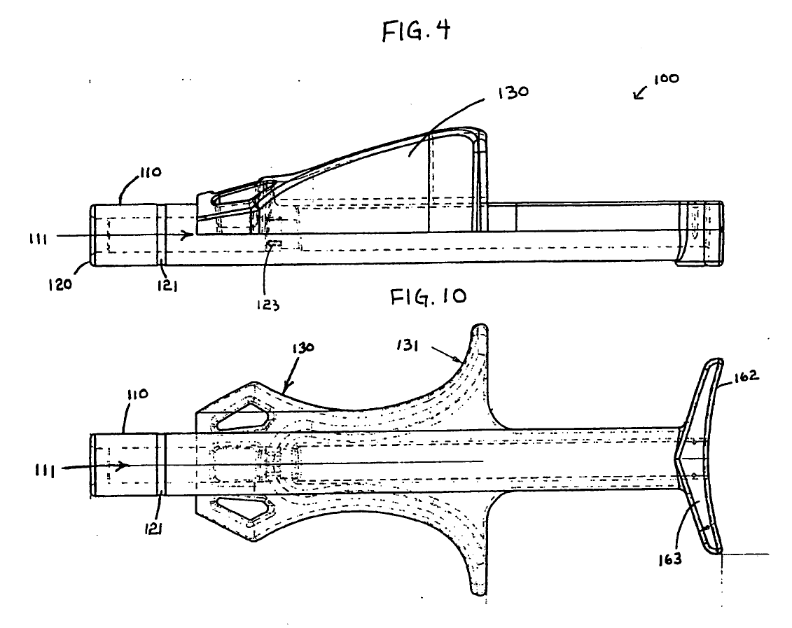

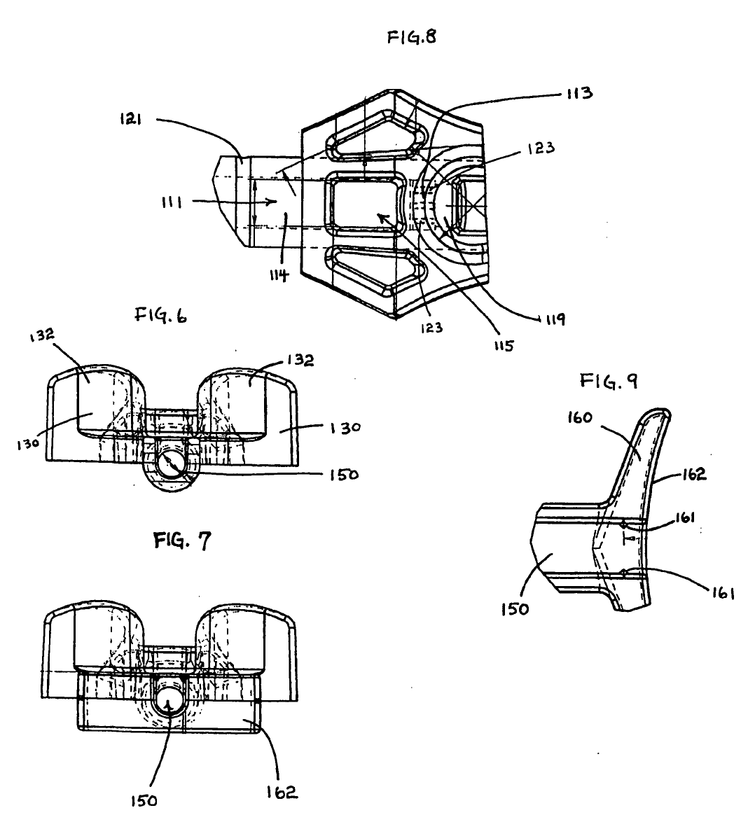

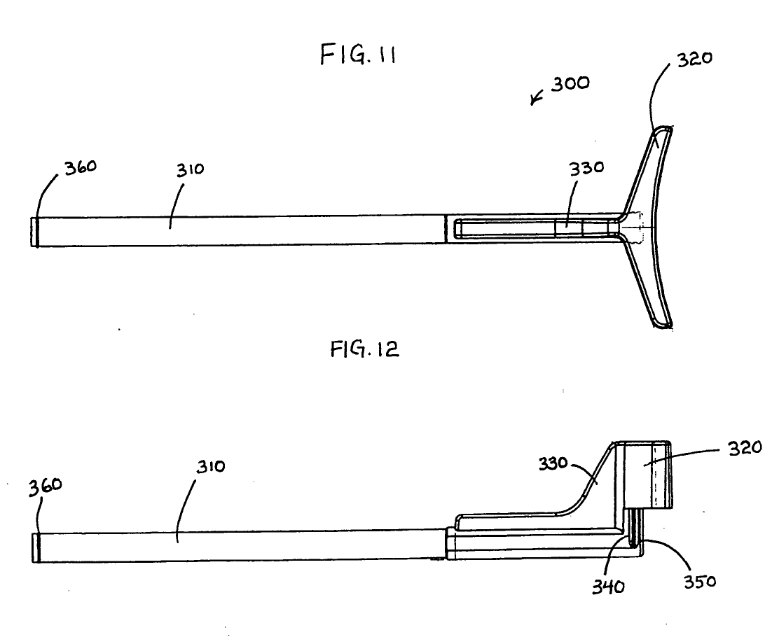

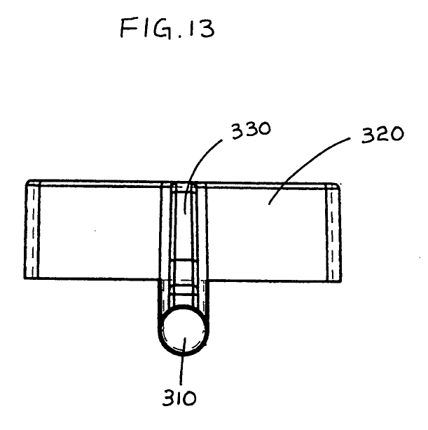

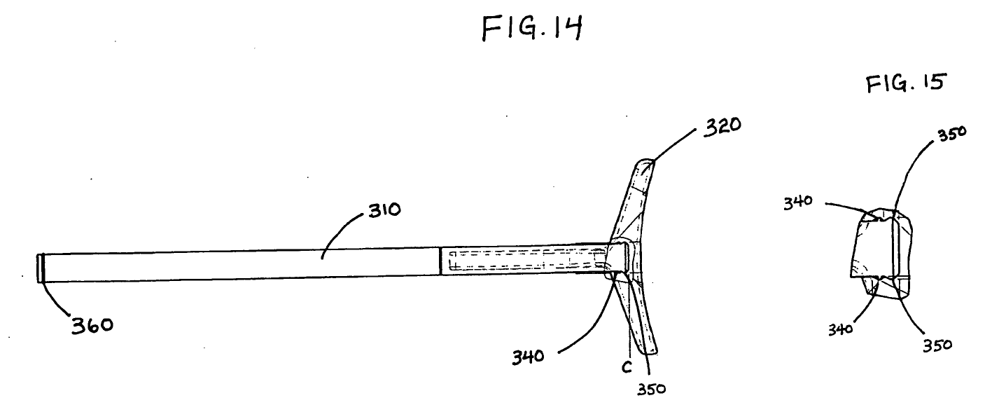

Reference is made to FIGS. 1 through 15, wherein an apparatus for implanting a marker constructed according to an embodiment of the invention is illustrated. FIGS. 1 through 3 show an implanter 10 having a housing 100, a cannula 200 and a pushrod 300.

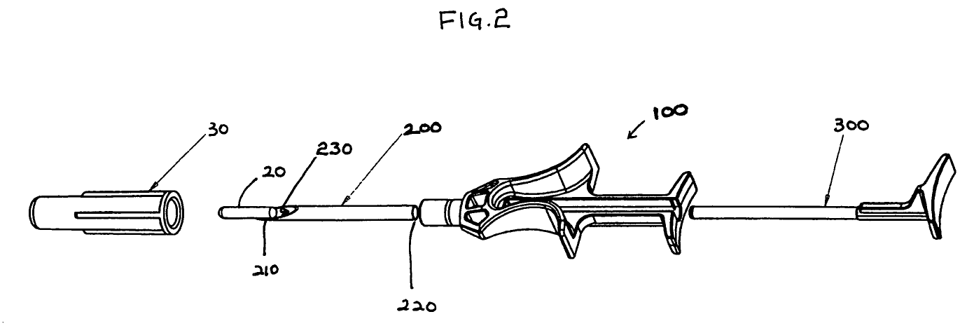

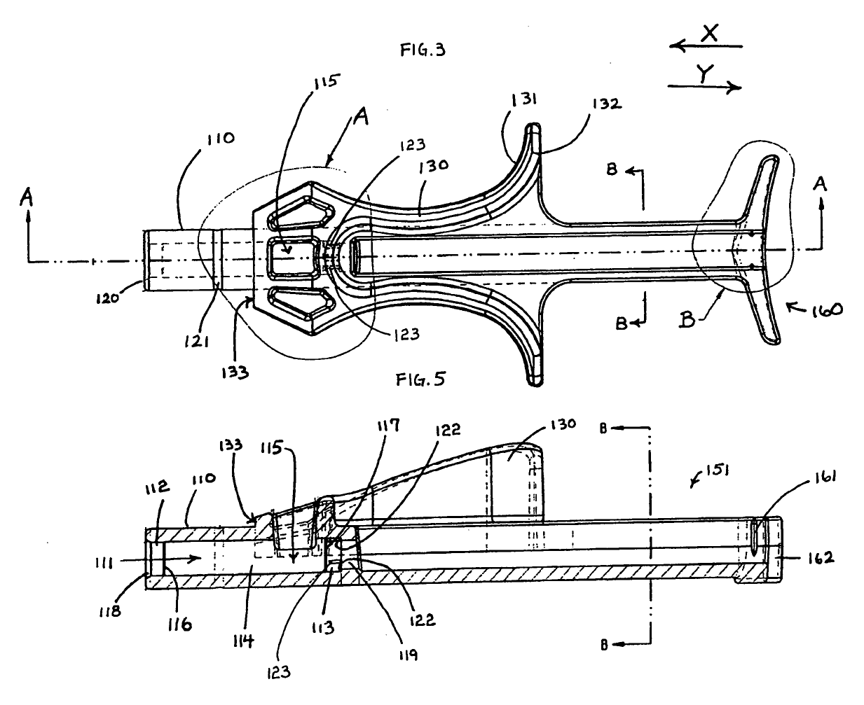

As shown in more detail in FIG. 2, an embodiment of cannula 200 of implanter 10 comprises a distal tip 210 and a proximal end 220, wherein the distal tip 210 is preferably angled in order to be substantially sharp enough to pierce the skin of an animal. Proximal end 220 is preferably blunt and is preferably inserted into housing 100 via bore 111, an embodiment of which is shown in FIGS. 3 through 5.

An embodiment of housing 100 is shown in FIGS. 3 through 10. As shown in detail in FIGS. 3 through 5, housing 100 preferably comprises a tubular member 110 having a bore 111 through which proximal end 220 of cannula 200 can be inserted. Bore 111 can comprise a cannula entrance 118 having a wider opening tapering to a distal portion 112 of bore 111, wherein the wider opening of cannula entrance 118 preferably facilitates the insertion of proximal end 220 of cannula 200. Cannula 200 is preferably inserted into bore 111 of housing 100 until proximal end 220 of cannula 200 contacts tubular shoulder 122, whereupon cannula 200 is prevented from being inserted any further. Cannula bore 230 is preferably aligned with a marker entrance 119 of tubular member 110, collectively creating a pathway for a marker 20 to pass.

Bore 111 preferably comprises distal portion 112 and a proximal portion 113, wherein distal portion 112 is further away from the user than proximal portion 113 when implanter 10 is properly held by the user. As shown in FIG. 5, an adhesive receiving space 114 is preferably located between distal portion 112 and proximal portion 113 of bore 111. According to the embodiment of tubular member 110 shown in FIG. 5, a distal ring 116 indicates the boundary between distal portion 112 and adhesive receiving space 114. Similarly, a proximal ring 117 can serve as the boundary between proximal portion 113 and adhesive receiving space 114. Preferably, distal ring 116 and proximal ring 117 each has a smaller inner diameter than adhesive receiving space 114 and more preferably engages cannula 200 when cannula 200 is inserted into tubular member 110 of housing 100. Most preferably, distal ring 116 and proximal ring 117 each engages cannula 200 and assist in aligning cannula bore 230 with marker entrance 119. Accordingly, when an adhesive is introduced into adhesive receiving space 114, distal ring 116 and proximal ring 117 can preferably contain the adhesive within adhesive receiving space 114. A preferred adhesive includes bonding compounds, more preferably a UV curing bonding compound.

Most preferably, as shown more clearly in FIG. 8, proximal portion 113 of bore 111 has a smaller diameter than adhesive receiving space 114, which can preferably contribute to maintaining the adhesive within adhesive receiving space 114 and substantially preventing the adhesive from entering marker entrance 119. Marker entrance 119 is preferably just wide enough to permit marker 20 to pass through into bore 11, while providing sufficient resistance therewith in order to prevent marker 20 from slipping out of marker entrance 119 once inserted. Accordingly, it is desirable to substantially prevent the adhesive from affecting the inner diameter of marker entrance 119 which can hinder marker 20 from being inserted.

Alternatively, adhesive receiving space 114 can be defined by the amount of adhesive inserted into bore 111 of housing 100. A preferred embodiment of the invention includes a plurality of ribs 123 in bore 111 which preferably assist in aligning cannula 200 concentrically with bore 111. More preferably, bore 111 comprises about three ribs 123.

In accordance with a preferred embodiment of the invention, an effective amount of an adhesive is inserted into adhesive receiving space 114 through an adhesive aperture 115 which preferably extends through a gripping structure 130 into adhesive receiving space 114 of bore 111. Preferably, the adhesive is inserted after cannula 200 is inserted into housing 100 and most preferably, while cannula 200 is mechanically pushed against shoulder 122, which can help maintain cannula 200 connected to marker entrance 119 and prevent the adhesive from flowing into marker entrance 119. The inserted adhesive preferably fills the area formed inside adhesive receiving space 114 and outside cannula 200. The adhesive preferably surrounds the outer circumference of cannula 200, and extends within adhesive receiving space 114 until the adhesive substantially fills adhesive receiving space 114, thereby securing cannula 200 to tubular member 110 of housing 100. Most preferably, as discussed above, distal ring 116 and proximal ring 117 prevent the adhesive from extending beyond distal ring 116 and proximal ring 117, thereby containing the adhesive to a concentrated area and preventing the adhesive from exiting tubular member 110.

Referring to FIGS. 3 and 10, an embodiment of housing 100 comprises a gripping structure 130. Gripping structure 130 can include a portion which extends over tubular member 110, wherein adhesive aperture 115 can extend through gripping structure 130 to adhesive receiving space 114 of bore 111, and can further include a portion which extend over an elongated member 151. Gripping structure 130 preferably further includes one or more, most preferably two, gripping surfaces 131 and one or more, most preferably two, stopping surfaces 132. Elongated member 151 preferably comprises a channel 150 and extends between tubular member 110 and a proximal member 160.

Proximal member 160 preferably includes a thumb support 162 beneficially positioned relative to gripping surface 131, the combination of which preferably provides a relatively stable and easy handling of implanter 10. For example, the user's index and middle fingers can engage one gripping surface 131 each, and the user's thumb can engage thumb support 162, thereby creating a stable handle on implanter 10 when the fingers and the thumb apply opposing forces, more specifically, in direction Y on gripping surfaces 131 and direction X on thumb support 162, respectively. Most preferably, gripping surface 131 and thumb support 162 comprise concave shapes for ergonomically engaging a finger or thumb.

Channel 150 preferably can receive marker 20 and permits marker 20 to move within the length of channel 150. Channel 150 preferably can also receive a portion of a pushrod 300, more preferably a rod 310 of pushrod 300, which preferably can be used to facilitate the insertion of marker 20 into cannula 200 and also to facilitate the ejection of marker 20 out of cannula 200 into the animal. An embodiment of pushrod 300 is illustrated in FIGS. 11 through 13. When inserting marker 20 into cannula 200, rod 310 of pushrod 300 is preferably located more proximal to the user than marker 20, thereby pushing marker 20 into cannula bore 230 through marker entrance 119 when the user applies a pushing force on a tail 320 of pushrod 300 in direction X, as shown in FIG. 3. When ejecting marker 20 from cannula 200 into the animal, a pushing force in direction X is preferably applied on tail 320 of pushrod 300, and therefore rod 310 contacts and applies a pushing force in direction X on marker 20 toward distal tip 210 of cannula 200 until marker 20 is ejected into the animal. Most preferably, tail 320 of pushrod 300 contacts one or more stopping surfaces 132 of gripping structure 130, which preferably prevents pushrod 300 from advancing any further in direction X. Additionally, a pushing force is preferably applied on tail 320 by a thumb, and tail 320 preferably comprises a concave surface for ergonomically engaging the thumb.

In accordance with an embodiment of the invention, pushrod 300 preferably comprises a circumferential pushrod rib 360 which preferably engages the inner surface of cannula bore 230, thereby creating a barrier packaging between cannula bore 230 on the proximal side of marker 20. As defined herein, a barrier packaging permits sterilizing agents in during sterilization and prevents the free movement of air in or out in ambient, thereby assisting in maintaining sterility. Therefore, the sterility of implanter 10 can be maintained until cover pushrod 300 is removed.

In accordance with a preferred embodiment of the invention, pushrod 300 comprises one or more proximal stoppers 350 and one or more corresponding distal stoppers. Proximal member 161 of housing 100 preferably comprises one or more corresponding ribs 161. Preferably, prior to the implantation procedure, pushrod 300 and housing 100 are arranged in a locked position so that ribs 161 of housing 100 are positioned between distal stoppers 340 and proximal stoppers 350. Therefore, pushrod 300 is preferably maintained in place and is substantially prevented from sliding in or out of channel 150. Preferably, pushrod 300 can only be released from this locked position with sufficient force, thereby substantially reducing the risk of removing pushrod from the locked position unintentionally. Accordingly, the risk of unintentionally ejecting marker 20 and the risk of unintentionally losing sterility are substantially reduced as well.

When ejecting marker 20 from cannula 200, pushrod 300 can be lifted off proximal member 160 to avoid contact between proximal stoppers 350 and ribs 161, and preferably is pushed in direction X with sufficient force to overcome proximal stoppers 350, whereupon pushrod 300 can be pushed in direction X until tail 320 contacts stopping surface 132 which preferably prevents pushrod 300 from advancing any further in direction X. The distance from distal tip 210 of cannula 200 traveled by marker 20 can therefore be known and standardized, and substantially precise positioning of marker 20 within the animal can be facilitated.

An embodiment of a cover 30 is shown in FIGS. 2, 16 and 17. Cover 30 preferably protects cannula 200, more specifically, distal tip 210 of cannula 200, by providing a barrier surrounding the portion of cannula 200 extending outside housing 100. Cover 30 preferably does not contact cannula 200, but engages housing 100, thereby substantially eliminating the risk of damaging distal tip 210 of cannula 200 during the application or removal of cover 30 onto implanter 10.

Cover 30 preferably comprises a cover bore 35 into which distal tip 210 of cannula 200 of implanter 10 can be inserted until a cover end surface 33 of a cover end 32 contacts a shoulder 133 of gripping structure 130, which preferably prevents further insertion of implanter 10 into cover 30. In accordance with a preferred embodiment of the invention, tubular member 110 of housing 100 comprises a distal end 120 having a rounded edge, thereby facilitating the insertion of tubular member 110 of housing 100 into cover 30. When cover end surface 33 engages shoulder 133, a cover rib 31 of cover 30 preferably engages a groove 121 of housing 100, thereby substantially maintaining cover 30 in position with respect to housing 100.

Preferably, cover rib 31 and groove 121 provide a barrier packaging between cover 30 and implanter 10 thereby maintaining the sterility of cannula 200 and the contents therein. Most preferably, as discussed above, rod 310 of pushrod 300 comprises a pushrod rib 360 which engages the inner surface of cannula bore 230. Accordingly, the area between cover rib 31 and pushrod rib 360 and its contents can preferably be sterilized with both cover 30 and rod 310 in place, and can maintain sterility until either cover 30 or pushrod 300 is removed. Preferably, cover 30 is removed immediately prior to implantation, thereby maintaining the sterility of cannula 200 and marker 20 until immediately prior to implantation. Whereas various methods and chemicals can be used to sterilize implanter 10, it is preferable to sterilize implanter 10 using ethylene oxide.

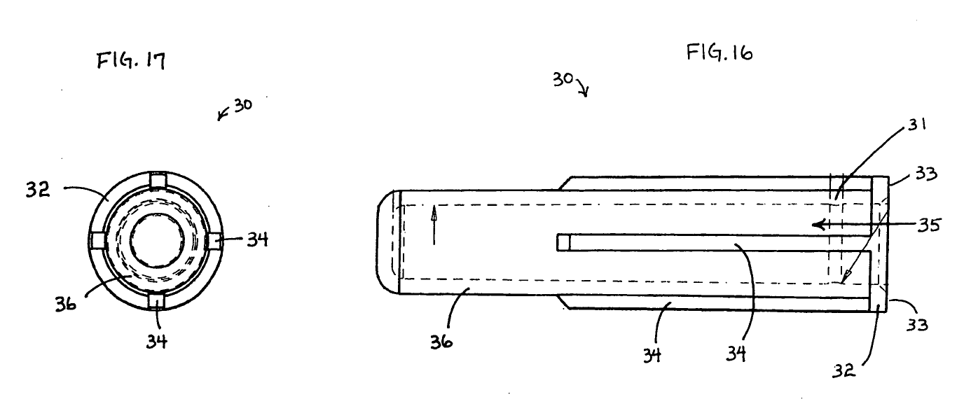

According to an embodiment of cover 30 shown in FIGS. 16-17, cover 30 preferably comprises a cover tube portion 36 connected to cover end 32, wherein cover end 32 has a larger circumference than cover tube portion 36. The embodiment of cover 30 further includes one or more cover support projections 34, which preferably strengthens cover 30 against external forces. It is to be understood that cover 30 can comprise a different shape or form, include additional features or exclude features described herein, without deviating from the scope of the invention, as a matter of application specific to design choice.

In production, the individual pieces of the embodiment of implanter 10 shown in FIGS. 1-15, more specifically, housing 100, cannula 200 and pushrod 300, are preferably produced independently of each other and then subsequently assembled to form implanter 10. For example, housing 100 can be injection molded or otherwise formed, preferably of a substantially rigid material, as a matter of application specific to design choice. Pushrod 300 can also likewise be injection molded, and preferably comprises polypropylene. Preferably, rod 310 is relatively flexible, which can reduce the risk of rod 310 breaking as well as facilitate its use. Cannula 200 preferably comprises stainless steel, and can be formed from a hollow stainless steel tube having a distal end and a proximal end. The distal end is preferably formed in the shape of an inclined edge having a sharp point, and the proximal end is preferably blunt.

In accordance with a preferred embodiment of the invention, the individually formed pieces of implanter 10 can be assembled in order to produce implanter 10. More specifically, cannula 200 can be inserted into bore 111 of tubular member 110 of housing 100 until proximal end 220 engages tubular shoulder 122. Preferably, housing 100 is molded with a plurality of ribs 123, more preferably about three ribs 123, on the inner surface of bore 111. Ribs 123 preferably assist in aligning cannula bore 230 with marker entrance 119. Once cannula 200 is in place, preferably while cannula is mechanically pushed against shoulder 122, an effective amount of an adhesive, preferably a UV curing bonding compound, is preferably inserted through adhesive aperture 115 into adhesive receiving space 114 of housing 100 in order to secure cannula 200 to housing 100. Preferably, the adhesive will substantially fill the area inside adhesive receiving space 114 and outside cannula 200, thereby sealing the gap between cannula 200 and housing 100 with the adhesive. Therefore, a substantially permanent bond is preferably created between housing 100 and cannula 200.

According to a preferred embodiment of the invention, an automated system is utilized to insert cannula 200 into housing 100, and preferably, the injection of the adhesive. Whereas the components can be manually assembled without deviating from the scope of the invention, an automated, such as a system robot and laser guide system, can substantially reduce the probability of error and misalignment and can reduce the potential risk of damaging the sharp distal tip 210 of cannula 200. A robotic system can also improve the efficiency in assembling the two components by preventing distal tip 210 of cannula 200 from contacting any object, thereby substantially reducing the risk of damaging distal tip 210 of cannula 200.

Marker 20 can be inserted into channel 150 and pushed by pushrod 300 into a desirable position within cannula 200. Implanter 10 having marker 20 inserted therein can thereafter be sterilized, preferably with ethylene oxide, most preferably with cover 30 and pushrod 300 in place. In accordance with an embodiment of the invention as shown in FIGS. 1-17, cover 30 and pushrod 300 collectively form barrier packaging and assist in retaining marker 20 within housing 100 and maintaining its sterility. The assembly of implanter 10, marker 20 and cover 30 can be transported thereafter, providing the user with an implanter that is sterile and ready to use. Alternatively, implanter 10, marker 20 and cover 30 can be transported either individually or after assembly, depending on the needs of the user.

According to an embodiment of the invention, cover 30 is injection molded and comprises polyethylene, and most preferably is substantially clear and provides visualization of cannula 200.

In use, if the assembly was sterilized, or after sterilizing the assembly at the laboratory wherein the implantation is performed, the user can remove cover 30 and insert implanter 10 into the animal being studied, preferably until distal end 120 of tubular member 110 of housing 100 contacts the skin of the animal. The user can thereafter push pushrod 300 in direction X by exerting a pushing force on tail 320 of pushrod 300, which will preferably eject marker 20 from within cannula 200 into the tissue of the animal. The spent implanter can be removed from the animal and is preferably discarded or sterilized for subsequent use. Most preferably, the spent implanter is discarded after a single use.

The examples provided are merely exemplary, as a matter of application specific to design choice, and should not be construed to limit the scope of the invention in any way.

Thus, while there have been shown and described and pointed out novel features of the present invention as applied to preferred embodiments thereof, it will be understood that various omissions and substitutions and changes in the form and details of the disclosed invention may be made by those skilled in the art without departing from the spirit of the invention. For example, housing 100 can exclude gripping surface 131, alter the number of stopping surfaces 132, including excluding it entirely, without deviating from the scope of the invention as a matter of application specific to design choice. Housing 100, pushrod 300, cannula 200 and/or cover 30 can comprise different materials than those described without deviating from the scope of the invention. The location, size, shape, and any other physical features of adhesive receiving space 114 can also be changed without deviating from the scope of the invention, as a matter of application specific to design choice. Additionally, other alterations, as a way of non-limiting example, marker entrance 119 can comprise substantially the same diameter of proximal portion 113 of bore 111, proximal portion 113 and distal portion 112 of bore 111 can comprise a diameter greater than or substantially equivalent to proximal ring 117 and distal ring 116, rod 310 of pushrod 300 can comprise different diameters, rod 310 can be non-cylindrical, tubular member 110 may be non-cylindrical, bore 111 can be non-cylindrical, etc., as a matter of application specific to design choice, without deviating from the scope of the invention. It is the intention, therefore, to be limited only as indicated by the scope of the claims appended hereto.

It is also to be understood that the following claims are intended to cover all of the generic and specific features of the invention herein described and all statements of the scope of the invention which, as a matter of language, might be said to fall therebetween.

What is claimed is:

1. An apparatus for implanting an implant in an animal, the apparatus comprising:
a cannula having a first end and a second end, wherein the first end comprises a sharp edge;
a housing for receiving the second end of the cannula, said housing defining an adhesive receiving space; and
adhesive in the space so that the cannula and the housing are joined together by the adhesive therein.

2. The apparatus for implanting a marker of claim 1, wherein the housing comprises one or more ribs in the adhesive receiving space.

3. The apparatus for implanting a marker of claim 1, wherein the housing comprises one or more rings on an inner surface of the cannula receiving member, wherein the one or more rings border the adhesive receiving space.

4. The apparatus for implanting a marker of claim 1, wherein the housing comprises two rings on an inner surface of the cannula receiving member, wherein the two rings form a first border and a second border of the adhesive receiving space.

5. The apparatus for implanting a marker of claim 1, wherein the second end of the cannula is received in the cannula receiving member.

6. The apparatus for implanting a marker of claim 1, comprising adhesive in the adhesive receiving space.

7. The apparatus for implanting a marker of claim 1, comprising adhesive in the adhesive receiving space, wherein the adhesive surrounds the cannula.

8. The apparatus for implanting a marker of claim 1, comprising a curing compound in the adhesive receiving space.

9. The apparatus for implanting a marker of claim 1, comprising a UV curing bonding compound in the adhesive receiving space.

10. The apparatus for implanting a marker of claim 1, wherein the cannula and the housing are permanently affixed to each other.

11. The apparatus for implanting a marker of claim 1, comprising an aperture suitable for injecting an adhesive into the adhesive receiving space.

12. The apparatus for implanting a marker of claim 1, comprising a lock suitable for retaining a pushrod in a locked position.

13. The apparatus for implanting a marker of claim 1, comprising a lock comprising one or more ribs on a pushrod and one or more ribs on the housing suitable for retaining the pushrod in a locked position.

14. The apparatus for implanting a marker of claim 1, comprising at least one gripping structure suitable for engaging one or more fingers.

15. The apparatus for implanting a marker of claim 1, wherein the housing comprises a channel.

16. The apparatus for implanting a marker of claim 1, comprising a cover.

17. The apparatus for implanting a marker of claim 1, comprising a cover suitable for placing over at least the distal end of the cannula, wherein the cover permits sterilizing agents to enter.

18. The apparatus for implanting a marker of claim 1, comprising a cover comprising a rib, wherein the housing comprises a groove, wherein the rib and the groove collectively create a barrier packaging.

19. The apparatus for implanting a marker of claim 1 comprising a pushrod comprising a rib, wherein the rib engages an inner surface of the cannula to create a barrier packaging.

20. The apparatus for implanting a marker of claim 1, wherein the cannula comprises steel.

21. The apparatus for implanting a marker of claim 1 comprising a pushrod.

22. The apparatus for implanting a marker of claim 1 comprising a pushrod comprising polypropylene.

23. A method of producing an apparatus for implanting a marker in an animal, the method comprising:
forming a cannula having a first end and a second end;
injection molding a housing comprising an adhesive receiving space;
inserting the second end into the housing;
introducing an adhesive in the adhesive receiving space for affixing a portion of the cannula to a portion of the housing.

24. The method of claim 23, comprising injection molding a pushrod.

25. The method of claim 23, wherein the housing comprises a tubular member having a bore wherein the second end of the cannula is inserted.

26. The method of claim 23, comprising creating a sharp edge on the first end of the cannula.

27. The method of claim 23, comprising inserting an adhesive into the adhesive receiving space.

28. The method of claim 23, comprising inserting a UV curing bonding compound into the adhesive receiving space.

29. The method of claim 23, comprising sterilizing the apparatus.

30. The method of claim 23, comprising applying sterilizing agents.

31. The method of claim 23, comprising applying sterilizing agents comprising ethylene oxide.

32. An apparatus for implanting an implant in an animal, the apparatus comprising a housing having at least a first end of a cannula inserted therein, wherein the apparatus comprises a UV curing bonding compound.

33. A method of producing an apparatus for implanting a marker in an animal, the method comprising inserting at least a portion of a cannula into a housing and introducing a UV curing bonding compound for affixing at least a portion of the cannula to the housing.
